(19) United States
(12) Patent Application Publication
(10) Pub. No.: US 2006/0064060 A1
(43) Pub. Date: Mar. 23, 2006

(54) Safety syringe with broken plunger

(76) Inventor: Hui-Chang Lin, Hemei Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/945,988

(22) Filed: Sep. 22, 2004

(51) Int. Cl. A61M 5/00; A61M 5/315

(52) U.S. Cl. 604/110; 604/220

(57) ABSTRACT

A syringe includes a barrel with a plunger movably inserted therein and a tube extends from a first end of the barrel and is in communication with a passage in the barrel. A flange extends inward from an inner periphery of the tube. A stopper is connected to the plunger and a rod extends from the plunger and through the stopper. The rod includes engaging protrusion which is engaged with the flange to prevent the plunger from being pulled back when the plunger is pushed into the barrel after injection. The rod includes a groove and the plunger can be broken at the groove.

### FIELD OF THE INVENTION

The present invention relates to a safety syringe with a plunger that can be broken after use so that the syringe cannot be re-used again.

### BACKGROUND OF THE INVENTION

A conventional hypodermic syringe generally includes a barrel with a locking tip on a first end thereof and a needle cannula is fixed to the locking tip. A plunger is movably received in the barrel so as to push the medicine in the barrel from the needle cannula. A protection cap is used to mount to the locking tip so as to receive the needle cannula in the cap. It will be a serious concern that the needle cannula could hurt the medical personnel during removing the protection cap from the locking tip before proceeding syringing and/or re-mounting the protection cap to the locking tip after the syringing process is finished. Besides, the discarded syringe could be re-used by some people who utilize the syringes, no matter whatever condition the syringes is, to inject drug into their bodies.

The present invention intends to provide a safety syringe including a plunger which has a head portion which is left and seals the tube of the barrel after use and the plunger is broken at the conjunction of the head portion.

### SUMMARY OF THE INVENTION

The present invention relates to a safety syringe that comprises a barrel with a passage defined therein and a tube extends from a first end of the barrel and is in communicating with the passage of the barrel. A flange extends inward from an inner periphery of the tube. A locking tip is connected to the tube and a cannula is connected to the locking tip. A plunger having a stopper connected to the first end thereof is inserted in the passage of the barrel and a rod extends from a first end of the plunger and through the stopper. The rod has an engaging protrusion extending radially from an outer periphery thereof so as to engage with the flange when the rod is inserted in the tube. The plunger can be broken at the groove defined in the outer periphery of the rod.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the syringe of the present invention;

FIG. 2 is a cross sectional view to show the syringe of the present invention;

FIG. 2-1 shows an enlarged view of the rod on the plunger;

FIG. 3A is a cross sectional view to show that the plunger is pushed toward the cannula;

FIG. 3B shows the rod on the plunger is engaged with the tube when fishing the injection;

FIG. 3C shows the plunger is broken at the groove;

FIG. 4 is an exploded view to show another embodiment of the syringe of the present invention;

FIG. 5 is a cross sectional view to show the syringe in FIG. 4;

FIG. 6A shows the rod on the plunger in FIG. 4 is engaged with the tube when fishing the injection, and

FIG. 6B shows the plunger is broken at the groove.

### DETAILED DESCRIPTION OF THE PREFERRED EMBODIMENT

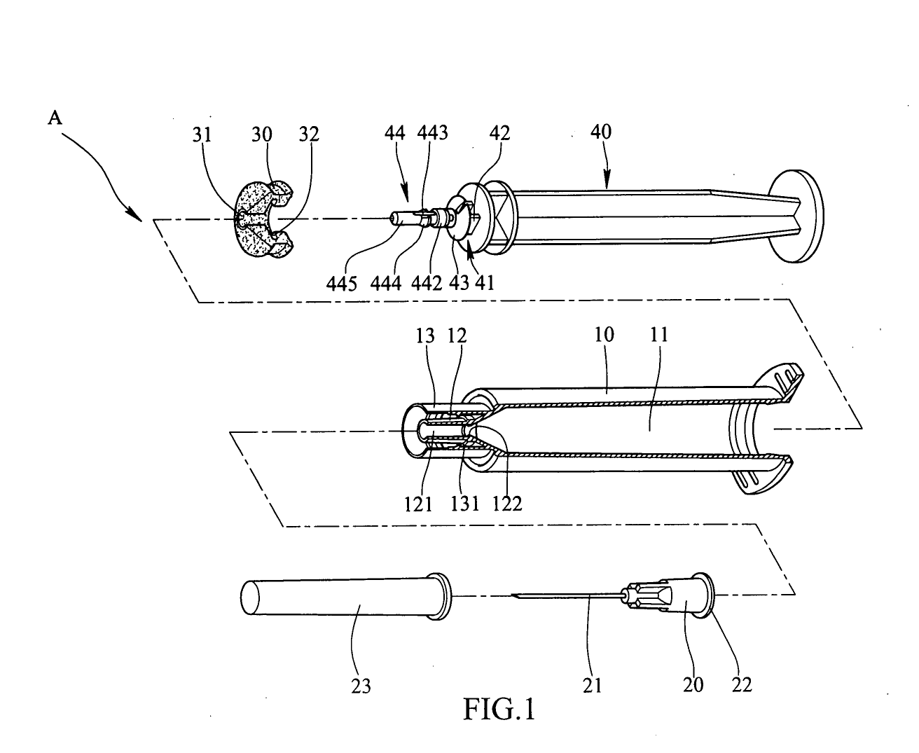

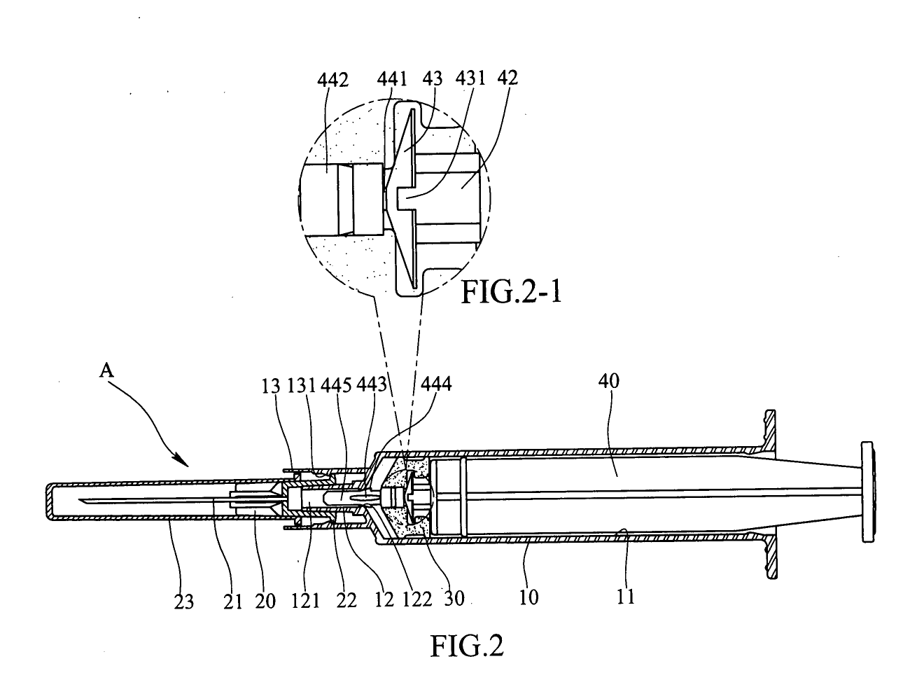

Referring to FIGS. 1, 2 and 2-1, the syringe “A” of the present invention comprises a barrel 10 with a passage 11 defined therein and a tube 12 extends from a first end of the barrel 10. The through passage 121 in the tube 12 is in communicating with the passage 11 of the barrel 10 and a flange 122 extends inward from an inner periphery of the tube 12. A tubular wall 13 extends from the first end of the barrel 10 and the tube 12 is enclosed by the tubular wall 13. A lip 131 extends inward from an inner periphery of the tubular wall 13. A locking tip 20 includes an positioning flange 22 at an end thereof and the positioning flange 22 is engaged with the lip 131 in the tubular wall 13. A cannula 21 is connected to the locking tip 20 and located at the end opposite to the end having the lip 131.

A plunger 40 has a stopper 30 connected to the first end thereof and the plunger 40 can be inserted in the passage 11 in the barrel 10 via a second end of the barrel 10. A connection portion 41 is connected the first end of the plunger 40 and includes ribs 42 and a skirt portion 43. A notch 431 is defined in the skirt portion 43 so as to provide the skirt portion 43 with suitable flexibility. A rod 44 extends from a center of the skirt portion 43 on the first end of the plunger 40. The stopper 30 includes a chamber with a receiving concavity 32 defined in an inner chamber thereof and a through hole 31 is defined through the stopper 30 and in communication with the chamber. The skirt portion 43 is received in the chamber and a periphery of the skirt portion 43 is engaged with the receiving concavity 32. The rod 44 extends through the through hole 31 of the stopper 30.

The rod 44 includes a base portion 442 and an extension portion 445, wherein the skirt portion 43 is connected to the base portion 442 and a groove 441 is defined in the outer periphery of the base portion 442 of the rod 44. A slot 443 is defined longitudinally through a wall of the extension portion 445 of the rod 44.

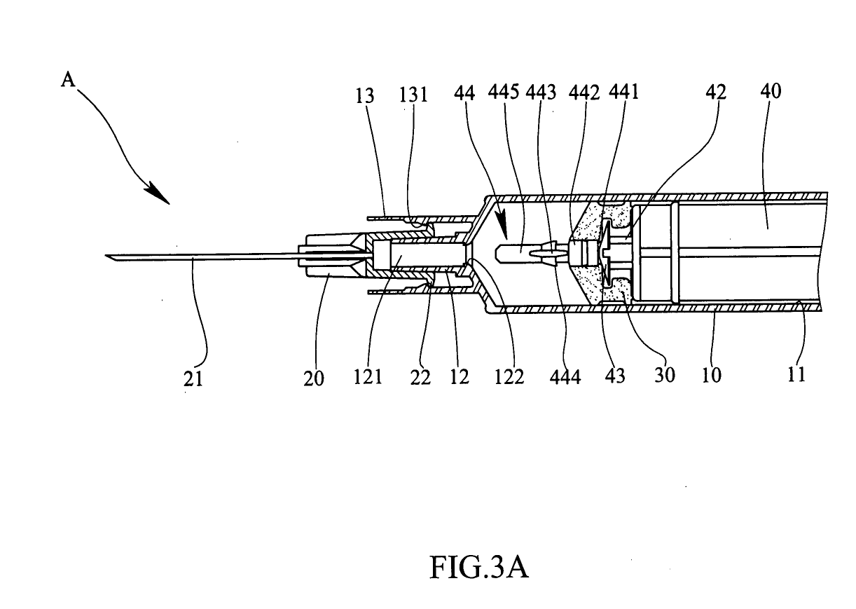

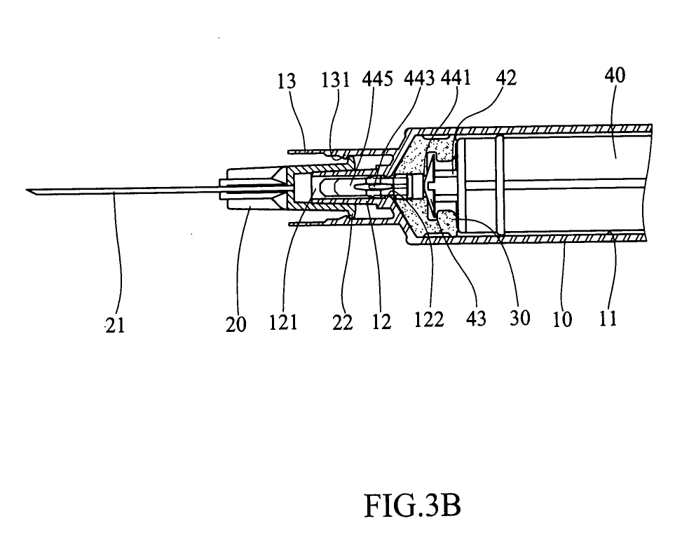

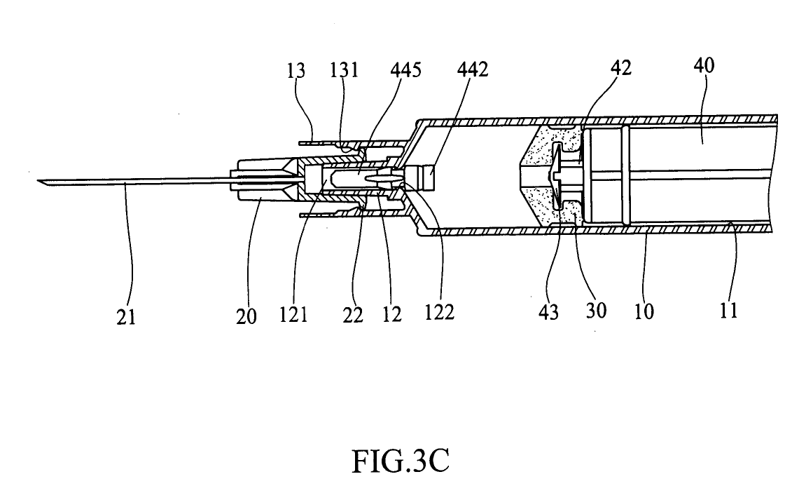

Further referring to FIGS. 3A, 3B and 3C, when operating the syringe, the plunger 40 is pushed toward the cannula 21 and the extension portion 445 is inserted into the tube 22 when finishing the injection. The extension portion 445 has an engaging protrusion 444 extending radially from an outer periphery thereof and the engaging protrusion 444 is engaged with the flange 122 when the extension portion 445 is completely inserted in the tube 12. By the engagement of the flange 122 and the engaging protrusion 444, the plunger 40 cannot be pulled backward. The user then applies a force to pull the plunger 40 which is broken at the groove 441.

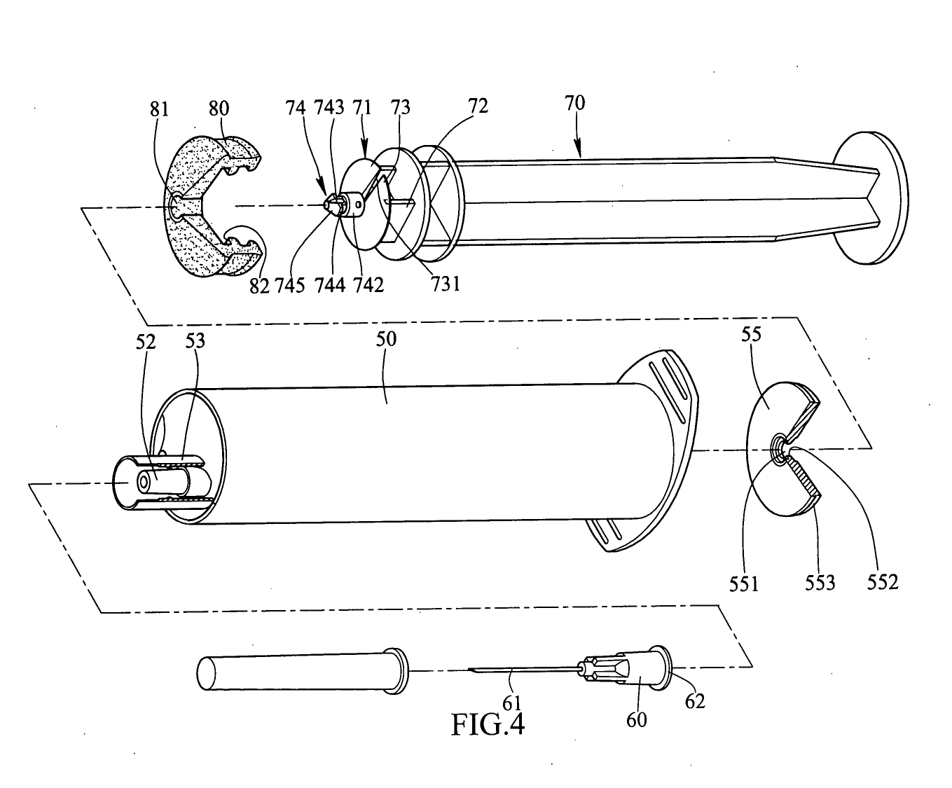

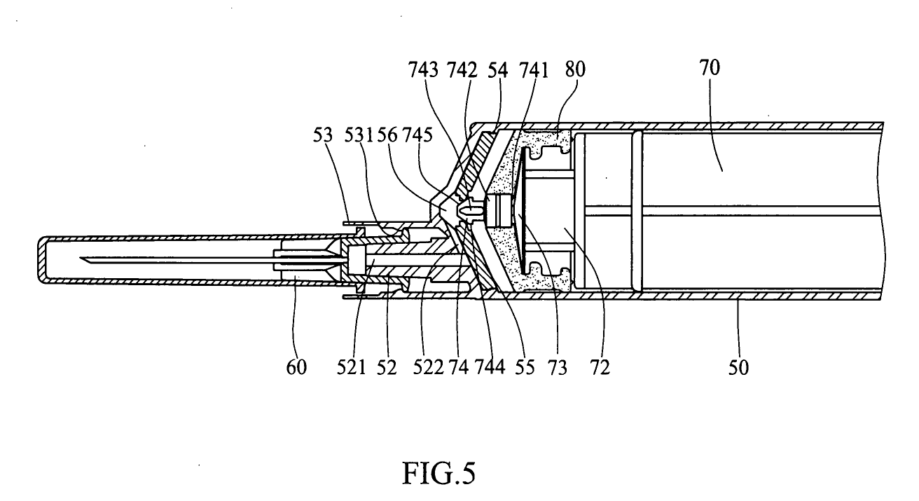

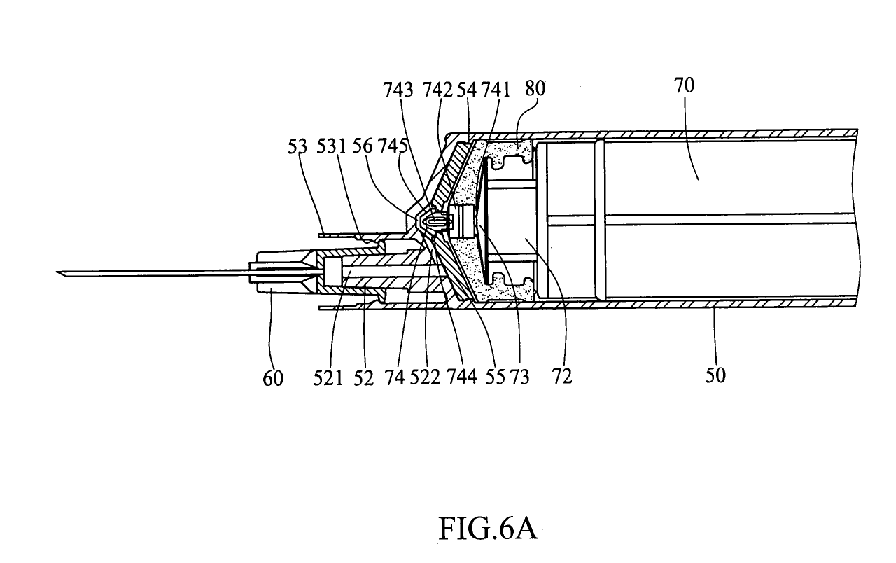

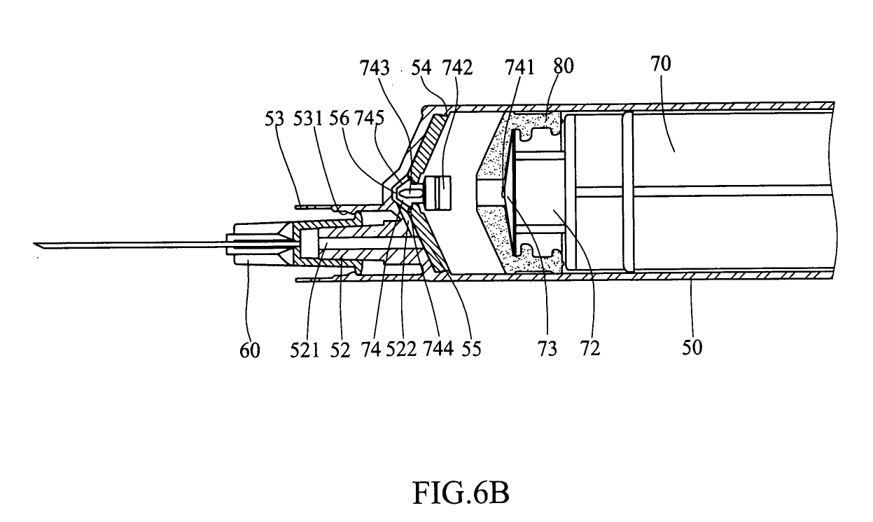

For a vein injection syringe, as disclosed in FIGS. 4, 5, 6A and 6B, the syringe includes a barrel 50 with a passage defined therein and a tube 52 extends eccentrically from a first end of the barrel 50. The tube 52 is in communicating with the passage of the barrel 50 and a tubular wall 53 extends from the first end of the barrel 50. The tube 52 is enclosed by a tubular wall 53 extends from the first end of the barrel 50 and a lip 531 extends inward from an inner periphery of the tubular wall 53 such that a locking tip 60 is connected to the tube 52 and an positioning flange 62 of the locking tip 60 is engaged with the lip 531 in the tubular wall 53. A cannula 61 is connected to the locking tip 60.

A collar 55 is engaged with an inside of the first end of the barrel 50 and has a hole 551 defined centrally therein and a flange 552 extends outward from an inner periphery of the hole 551.

A plunger 70 has a stopper 80 connected to the first end thereof and a rod 74 extends from a first end of the plunger 70 and through the stopper 80. The rod 74 includes a base portion 742 and an extension portion 745, an engaging protrusion 744 extends radially from an outer periphery of the extension portion 745 and a slot 743 is defined longitudinally through a wall of the rod 74. A connection portion 71 includes ribs 72 extending from the plunger 70 and a skirt portion 73 is connected to the ribs 72. The stopper 80 includes a chamber with a receiving concavity 82 defined in an inner chamber thereof. A through hole 81 is defined through the stopper 80 and in communication with the chamber. The skirt portion 73 includes a notch 731 and is received in the chamber and a periphery of the skirt portion 73 is engaged with the receiving concavity 82. The extension portion 745 the rod 74 extends through the through hole 81. A groove 741 is defined in the outer periphery of the extension portion 742 and located between the base portion 742 and the skirt portion 71.

When the plunger 70 is pushed to its limit position, the extension portion 745 extends through the hole 551 in the collar 55 and can be received in the space 56 defined between an inside of the barrel 50 and the collar 55, the engaging protrusion 744 is engaged with the flange 552 such that the plunger 70 cannot be pulled backward. A path 522 is defined between the inside of the first end of the barrel 50 and the collar 55 and is in communication with the tube 52 so that medicine can be pushed into the tube 52 via the path 522. The plunger 70 can then be broken at the groove 741.

While we have shown and described the embodiment in accordance with the present invention, it should be clear to those skilled in the art that further embodiments may be made without departing from the scope of the present invention.

What is claimed is:

1. A safety syringe comprising:
a barrel with a passage defined therein and a tube extending from a first end of the barrel, the tube being in communicating with the passage of the barrel, a flange extending inward from an inner periphery of the tube, a locking tip connected to the tube and a cannula connected to the locking tip, and
a plunger having a stopper connected to the first end thereof and a rod extending from a first end of the plunger and through the stopper, the rod having an engaging protrusion extending radially from an outer periphery thereof and a groove defined in the outer periphery of the rod, the engaging protrusion engaged with the flange when the rod is inserted in the tube and the plunger being capable of being broken at the groove.

2. The syringe as claimed in claim 1, wherein a tubular wall extends from the first end of the barrel and the tube is enclosed by the tubular wall, a lip extending inward from an inner periphery of the tubular wall, the locking tip including an positioning flange which is engaged with the lip in the tubular wall.

3. The syringe as claimed in claim 1 further comprising a slot defined longitudinally through a wall of the rod.

4. The syringe as claimed in claim 1 further comprising a skirt portion extending from the rod and the stopper including a chamber with a receiving concavity defined in an inner chamber thereof, a through hole defined through the stopper and in communication with the chamber, the skirt portion being received in the chamber and a periphery of the skirt portion engaged with the receiving concavity, the rod extending through the through hole.

5. The syringe as claimed in claim 4, wherein the skirt portion includes a notch defined therethrough so that the skirt portion is flexible.

6. A safety syringe comprising:
a barrel with a passage defined therein and a tube extending eccentrically from a first end of the barrel, the tube being in communicating with the passage of the barrel, a collar engaged with an inside of the first end of the barrel and having a hole defined centrally therein and a flange extending outward from an inner periphery of the hole, a locking tip connected to the tube and a cannula connected to the locking tip, and
a plunger having a stopper connected to the first end thereof and a rod extending from a first end of the plunger and through the stopper, the rod having an engaging protrusion extending radially from an outer periphery thereof and a groove defined in the outer periphery of the rod, the engaging protrusion engaged with the flange when the rod extends through the hole in the collar and the plunger being capable of being broken at the groove.

7. The syringe as claimed in claim 6, wherein a tubular wall extends from the first end of the barrel and the tube is enclosed by the tubular wall, a lip extending inward from an inner periphery of the tubular wall, the locking tip including an positioning flange which is engaged with the lip in the tubular wall.

8. The syringe as claimed in claim 6 further comprising a slot defined longitudinally through a wall of the rod.

9. The syringe as claimed in claim 6 further comprising a skirt portion extending from the rod and the stopper including a chamber with a receiving concavity defined in an inner chamber thereof, a through hole defined through the stopper and in communication with the chamber, the skirt portion being received in the chamber and a periphery of the skirt portion engaged with the receiving concavity, the rod extending through the through hole.

10. The syringe as claimed in claim 9, wherein the skirt portion includes a notch defined therethrough so that the skirt portion is flexible.
